(19) United States
(12) Patent Application Publication
(10) Pub. No.: US 2025/0152032 A1
(43) Pub. Date: May 15, 2025

(54) Medical Systems, Devices, and Kits Useful in Performing Treatment under Magnetic Resonance Imaging and Related Methods

(71) Applicants: Cook Medical Technologies LLC, Bloomington, IN (US); Muffin Incorporated dba Cook Advanced Technologies, West Lafayette, IN (US)

(72) Inventors: Eric D. Anttila, West Lafayette, IN (US); Grant M. Baker, Lafayette, IN (US); Eric Brandner, West Lafayette, IN (US); Sean D. Chambers, Bloomington, IN (US); David C. Gross, Lafayette, IN (US); Joshua F. Krieger, Topsfield, MA (US); Ram H. Paul, Bloomington, IN (US); Andrew J. Robison, Otterbein, IN (US); Jesse Roll, West Lafayette, IN (US)

(73) Assignees: Cook Medical Technologies LLC, Bloomington, IN (US); Muffin Incorporated dba Cook Advanced Technologies, West Lafayette, IN (US)

(21) Appl. No.: 18/836,258

(22) PCT Filed: Feb. 7, 2023
(86) PCT No.: PCT/US2023/062097

§ 371 Date: Aug. 6, 2024

Related U.S. Application Data
(60) Provisional application No. 63/307,356, filed on Feb. 7, 2022.

(51) Int. Cl. A61B 5/055

(52) U.S. Cl. CPC A61B 5/055

(57) ABSTRACT

Example medical systems useful in performing treatment under magnetic resonance imaging and related devices, kits, and methods are described. An example medical system includes a first medical device and a second medical device. The first medical device has a first passive marker. The first passive marker is formed of a first material that has a first magnetic susceptibility. The second medical device has a second passive marker. The second passive marker is formed of a second material that has a second magnetic susceptibility. The second passive marker creates an image artifact under MRI. The image artifact created by the second passive marker is altered under MRI when the first passive marker overlaps the second passive marker.

### FIELD

The disclosure relates generally to the field of medical devices. More particularly, the disclosure relates to medical systems useful in performing treatment under magnetic resonance imaging (MRI), medical devices useful in performing treatment under MRI, kits useful in performing treatment under MRI, and methods of performing treatment under MRI.

### BACKGROUND

The field of interventional MRI is gaining wider acceptance and seeing an increase in the number of procedures that can be performed. Interventional procedures conducted under MRI have several benefits over X-Ray-guided interventions. For example, the patient is not exposed to ionizing radiation. Also, MRI provides the ability to characterize tissue and functional flow during an interventional procedure.

The development of interventional procedures conducted under MRI has been limited as a result of the tools needed to perform these procedures being unavailable. Therefore, patients are required to make multiple visits to treatment facilities to visualize, diagnose, and treat various conditions. In addition, multiple imaging modalities are often needed, which impacts the accuracy of utilizing a magnetic resonance (MR) image in directing intervention. For example, interventional medical devices having a high magnetic susceptibility that are placed in magnetic fields generated by an MR scanner have the tendency to interact with and/or distort the external applied field. As a result, these devices can result in large image artifacts that can obliterate key features in targeted anatomy and can potentially harm a patient being scanned due to the forces and torques induced by this interaction. Alternatively, interventional medical devices that have a low magnetic susceptibility that are placed in magnetic fields generated by an MR scanner can render the devices nearly invisible under MRI, making the identification, tracking, and navigation of the devices nearly impossible during interventional procedures.

To overcome these challenges, current MRI-compatible devices have employed a variety of visualization techniques, including passive susceptibility-based markers, semi-active resonant circuits, and active antenna coils. Of these, passive susceptibility-based markers offer the fewest design and fabrication complications as they do not require any electric circuit design and integration or require high frequency operation. However, current passive susceptibility-based markers are typically tailored for a specific device, remain fixed in a particular configuration on a device or component, and produce image artifacts under MRI that cannot be altered during the performance of a procedure.

A need exists, therefore, for new and improved medical systems useful in performing treatment under MRI, medical devices useful in performing treatment under MRI, kits useful in performing treatment under MRI, and methods of performing treatment under MRI.

### SUMMARY OF SELECTED EXAMPLE EMBODIMENTS

Various example medical systems useful in performing treatment under MRI, medical devices useful in performing treatment under MRI, kits useful in performing treatment under MRI, and methods of performing treatment under MRI are described herein.

An example medical system useful in performing treatment under MRI includes a first medical device and a second medical device. The first medical device has a first passive marker. The first passive marker is formed of a first material that has a first magnetic susceptibility. The second medical device has a second passive marker. The second passive marker is formed of a second material that has a second magnetic susceptibility. The second passive marker creates an image artifact under MRI. The image artifact created by the second passive marker is altered under MRI when the first passive marker overlaps the second passive marker.

An example kit useful in performing treatment under MRI includes a medical system that includes a first medical device and a second medical device. The first medical device has a first passive marker. The first passive marker is formed of a first material that has a first magnetic susceptibility. The second medical device has a second passive marker. The second passive marker is formed of a second material that has a second magnetic susceptibility. The second passive marker creates an image artifact under MRI. The image artifact created by the second passive marker is altered under MRI when the first passive marker overlaps the second passive marker.

An example method of performing treatment under MRI comprises positioning a patient within a magnetic resonance scanner; scanning a first portion of the patient using the magnetic resonance scanner, the magnetic resonance scanner having a first set of MR image parameters; obtaining a magnetic resonance image of the first portion of the patient; identifying a tissue that has predefined characteristics using the magnetic resonance image; selecting a procedure to treat the tissue based upon the predefined characteristics; selecting a medical system that has a first medical device and a second medical device to accomplish performance of the procedure; while the patient remains positioned within the magnetic resonance scanner used to scan the first portion of the patient, advancing the medical system into a bodily passage and to the tissue while scanning a second portion of the patient that includes the medical system and an image artifact created by a marker disposed on the second medical device using the magnetic resonance scanner; obtaining a magnetic resonance image of the second portion of the patient that includes the medical system and the image artifact created by the marker; identifying the image artifact within the MR image; confirming the position of the medical system within the bodily passage; manipulating the position of the first medical device relative to the second medical device to alter the image artifact created by the marker by overlapping the marker with a second marker disposed on the first medical device; scanning a third portion of the patient that includes the medical system and an altered image artifact using the magnetic resonance scanner; obtaining a magnetic resonance image of the third portion of the patient that includes the medical system and the altered image artifact; altering the first set of MR image parameters to a second set of MR image parameters based upon the altered image artifact; and performing treatment.

Additional understanding of these example medical systems, devices, kits, and methods can be obtained by review of the detailed description, below, and the appended drawings.

### BRIEF DESCRIPTION OF THE DRAWINGS

FIG. 1 is a partial elevation view of a first example medical system that includes a first example medical device and a second example medical device.

FIG. 2 is an elevation view of the first example medical device included in the medical system illustrated in FIG. 1.

FIG. 3 is an elevation view of the second example medical device included in the medical system illustrated in FIG. 1.

FIG. 4 is a magnified view of area A in FIG. 2.

FIG. 5 is a magnified view of area B in FIG. 3.

FIG. 6 is an exploded elevation view of a second example medical system that includes a first example medical device and a second example medical device.

FIG. 7 is an exploded elevation view of a third example medical system that includes a first example medical device and a second example medical device.

FIG. 8 illustrates an example kit that includes example medical systems useful in performing treatment under MRI.

FIG. 9 is a partial perspective view of a fourth example medical system that includes a first example medical device, a second example medical device, a third example medical device, and a fourth example medical device.

FIG. 10 is a cross-sectional view of the fourth example medical system shown in FIG. 9 taken along line 10-10.

FIG. 11 is a schematic illustration of an example method of performing treatment under MRI.

FIG. 12 is an MR image showing a first medical device of a medical system disposed within a bodily passage. The first medical device includes a first plurality of markers. Each marker produces an image artifact under MRI.

FIG. 13 is an MR image showing a medical system disposed within a bodily passage. The medical system includes a first medical device and a second medical. The first medical device includes a first plurality of markers and the second medical device includes a second plurality of markers. The overlapping arrangement of the first and second plurality of markers alters (e.g., amplifies, attenuates, eliminates) the image artifact produced by the first plurality of markers under MRI.

FIG. 14 is an MR image showing a medical system disposed within a bodily passage. The medical system includes a first medical device and a second medical device. The first medical device includes a first plurality of markers and the second medical device includes a second plurality of markers. The overlapping arrangement of the first and second plurality of markers alters (e.g., amplifies) the image artifact produced by the first plurality of markers under MRI.

FIG. 15 is an MR image showing a medical system disposed within a bodily passage. The medical system includes first medical device and a second medical device. The first medical device includes a first plurality of markers and the second medical device includes a second plurality of markers. The overlapping arrangement of a marker of the first plurality of markers and a marker of the second plurality of markers alters (e.g., amplifies, attenuates, eliminates) the image artifact produced by the marker of the first plurality of markers under MRI.

### DETAILED DESCRIPTION OF SELECTED EXAMPLES

The following detailed description and the appended drawings describe and illustrate various example medical systems useful in performing treatment under MRI, medical devices useful in performing treatment under MRI, kits useful in performing treatment under MRI, and methods of performing treatment under MRI. The description and illustration of these examples are provided to enable one skilled in the art to make and use a medical system, device, a kit, and to practice a method of performing treatment under MRI. They are not intended to limit the scope of the invention, or the protection sought, in any manner. The invention is capable of being practiced or carried out in various ways and the examples described and illustrated herein are merely selected examples of the various ways of practicing or carrying out the invention and are not considered exhaustive.

As used herein, the term “attached” refers to one member being secured to another member such that the members do not completely separate from each other during use performed in accordance with the intended use of an item that includes the members in their attached form.

As used herein, the term “circumference” refers to an external, or internal, enclosing boundary of a body, element, or feature and does not impart any structural configuration on the body, element, or feature.

As used herein, the term “marker” refers to a discrete deposit of a first material on a second material such that the first material is visible under MRI and is distinguishable from the second material under MRI, a portion of an interventional device in which a first material has been incorporated into a second material such that the combination of the first and second materials is visible under MRI and is distinguishable from the second material under MRI, and/or a portion of an interventional device in which a material that forms a portion of an interventional device has been manipulated such that the portion is visible under MRI and is distinguishable from the remainder of the interventional device under MRI.

As used herein, the term “passive,” in relation to a marker, refers to a marker that is either unpowered or powered exclusively by the electromagnetic field of a MR scanner.

As used herein, the term “treatment” refers to a medical procedure performed on or in a portion of a body of a patient. Examples of treatments include delivery of an agent to a site within a body vessel, modification of a local environment inside of a body vessel such as by heating or cooling, and removal of a tissue or portion of a tissue from a site within a body of a patient (i.e., biopsy).

As used herein, the term “magnetic susceptibility” refers to the intrinsic property of a material that relates to how much the material will become magnetized in an applied magnetic field.

As used herein, the term “susceptibility,” without “magnetic,” refers to the ability of an element to influence an external magnetic field. Susceptibility is dependent on various properties of an element, including the size, density, volume, geometric configuration, and other physical properties, and the magnetic susceptibility of the material of which the element is formed.

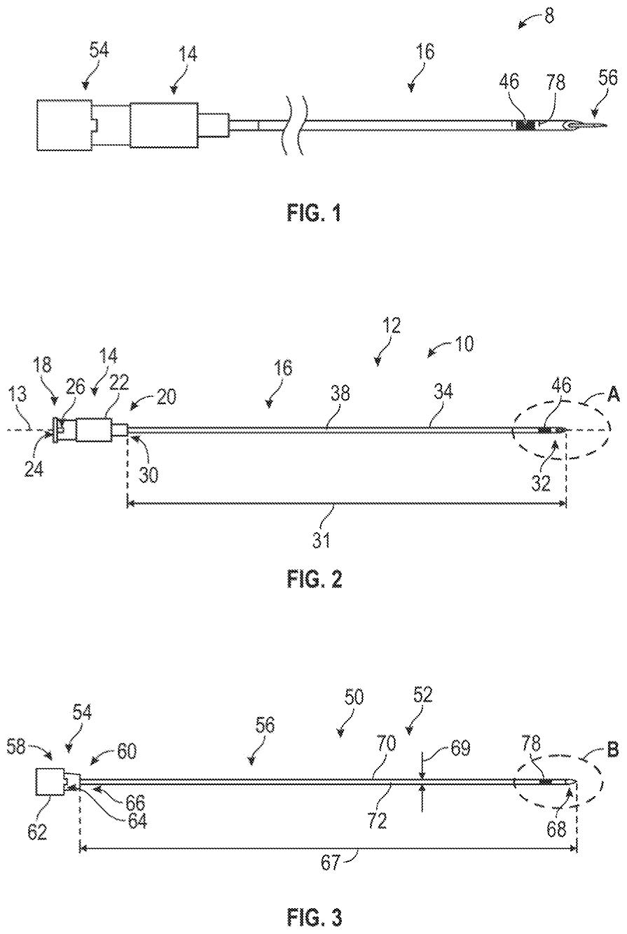

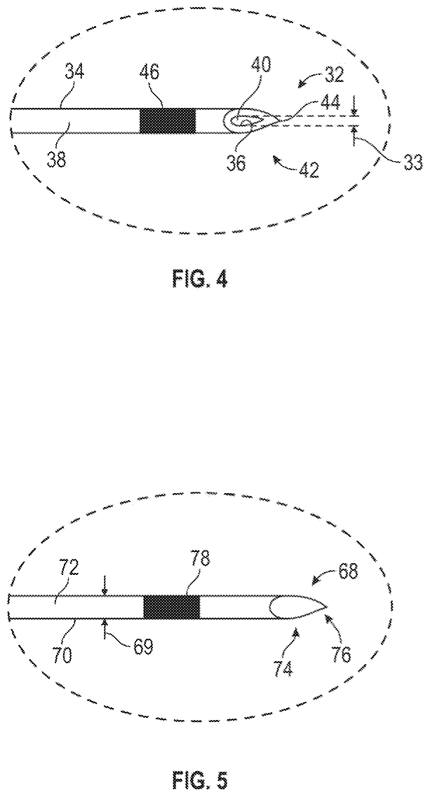

FIGS. 1, 2, 3, 4, and 5 illustrate a first example medical system 8 useful in performing treatment under MRI. In this example, the first example medical system 8 includes a first medical device 10 and a second medical device 50.

In the illustrated embodiment, the first medical device 10 is a cannula 12 and the second medical device 50 is a stylet 52 that can be used coaxially with the first medical device 10.

The cannula 12 has a lengthwise axis 13, a hub member 14 and an elongate member 16. The hub member 14 has a proximal end 18, a distal end 20, and a main body 22 that defines a lumen 24 and a projection 26. The hub member 14 provides a mechanism for attaching other devices to the cannula 12 (e.g., second medical device 50). The lumen 24 extends from the proximal end 18 to the distal end 20 such that one or more devices can be passed into, and through, the hub member 14. The elongate member 16 has a proximal end 30, a distal end 32, a length 31, an inside diameter 33, an outer surface 34, inner surface 36, and a main body 38 that defines a lumen 40 and a distal tapered tip 42 with a cutting edge 44. The lumen 40 extends from the proximal end 30 to the distal end 32 such that one or more devices can be passed into, and through, the elongate member 16 of the cannula 12. In the illustrated embodiment, the elongate member 16 of the cannula 12 is formed of a first material that has a first magnetic susceptibility.

The elongate member 16 of the cannula 12 includes a first marker 46 disposed between the proximal end 30 and the distal end 32 of the elongate member 16 and around the entire circumference of the main body 38 of the elongate member 16 of the cannula 12. The first marker 46 is a passive marker and is formed of a second material (e.g., pure titanium) that is different than the first material. The second material has a second magnetic susceptibility that is different than the first magnetic susceptibility and creates an image artifact under MRI that has first set of characteristics (e.g., size, shape, position, orientation, magnetic susceptibility, intensity).

The stylet 52 has a hub member 54 and an elongate member 56. The hub member 54 has a proximal end 58, a distal end 60, and a main body 62 that defines a recess 64. The hub member 54 provides a mechanism for attaching other devices to the stylet 52 (e.g., first medical device 10). The recess 64 extends into the main body 62 from a location between the proximal end 58 and the distal end 60 and toward the proximal end 58. The recess 64 is sized to receive the projection 26 defined by the hub member 14 of the cannula 12 such that releasable attachment between the cannula 12 and the stylet 42 can be accomplished. The elongate member 56 has a proximal end 66, a distal end 68, a length 67, an outside diameter 69, an outer surface 70, and a main body 72 that defines a solid member with a distal tapered tip 74 with a cutting edge 76. The length 67 of the elongate member 56 of the stylet 52 is greater than the length 31 of the elongate member 16 of the cannula 12. The outside diameter 69 of the elongate member 56 of the stylet 52 is less than the inside diameter 33 of the elongate member 16 of the cannula 12. This structural arrangement allows for the elongate member 56 of the stylet 52 to be passed into, and through, the lumen 24 of the hub member 14 and the lumen 40 of the elongate member 16 of the cannula 12, as shown in FIG. 1. The elongate member 56 of the stylet 52 is formed of a third material that has a third magnetic susceptibility. In the illustrated embodiment, the first material that forms the elongate member 16 of the cannula 12 and the third material that forms the elongate member 56 of the stylet 52 are the same.

In the embodiment illustrated, the elongate member 56 of the stylet 52 includes a second marker 78 disposed between the proximal end 66 and the distal end 68 and around the entire circumference of the main body 62 of the elongate member 56 of the stylet 52. The second marker 78 is a passive marker and is formed of a fourth material (e.g., polycrystalline graphite) that is different than the first material and the third material and that has a fourth magnetic susceptibility that is different than (e.g., greater than, less than, opposing) the first magnetic susceptibility and the third magnetically susceptibility. The second marker 78 creates an image artifact under MRI that has a second set of characteristics (e.g., size, shape, position, orientation, magnetic susceptibility, intensity) that are different than the first set of characteristics. The second marker 78 is positioned on the elongate member 56 such that the first marker 46 can overlap the second marker 78 when the first and second medical devices 10, 50 are used in combination. For example, as shown in FIG. 1, the first and second markers 46, 78 overlap when the second medical device 50 is disposed within the first medical device 10 and the first and second markers 46, 78 are disposed at the same location (e.g., axially) along the lengthwise axis 13 of the elongate member 16 of the cannula 12.

In the illustrated embodiment, each of the markers 46, 78 is a band of material attached to their respective elongate members 16, 56. Each of the first and third materials is a non-magnetically susceptible material, or a material that has a low magnetic susceptibility (e.g., non-paramagnetic material, a non-ferromagnetic material, a non-diamagnetic material) relative to the magnetic susceptibility of a material that forms a marker included on a medical device, and the material used to form a marker included on a medical device is a magnetically susceptible material, or a material that has a high magnetic susceptibility (e.g., a paramagnetic material, a ferromagnetic material, a diamagnetic material) relative to the magnetic susceptibility of a material that forms another portion of a medical device (e.g., elongate member), or a material that has a low magnetic susceptibility (e.g., a non-paramagnetic material, anon-ferromagnetic material, a non-diamagnetic material) relative to the magnetic susceptibility of a material that forms another portion of a medical device (e.g., elongate member) and/or a marker.

The first material that forms the elongate member 16 of the first medical device 10, the second material that forms the elongate member 56 of the second medical device 50, and the material that forms a marker included on a medical device can comprise any suitable material with the magnetic susceptibility described herein. For example, the first material, the second material, the third material, and the fourth material can comprise any suitable MRI compatible material having the magnetic susceptibility described herein. In the embodiment illustrated, each of the first material and the third material comprises titanium and the first marker 46 is formed of second material that has a second magnetic susceptibility that alters an image artifact created by the second marker 78 under MRI when the first marker 46 overlaps (e.g., axially) the second marker 78. Alternatively, a second marker can be formed of a fourth material that has a fourth magnetic susceptibility that alters an image artifact created by a first marker under MRI when the first marker overlaps (e.g., axially) the second marker during use. These alternatives can be accomplished by forming a first marker and a second marker of the same materials or different materials, or of materials that have different magnetic susceptibilities. When formed of the same materials, the second magnetic susceptibility, or second susceptibility, can be the same as, or different than, the fourth magnetic susceptibility, or fourth susceptibility, depending on various considerations, such as how a material is applied to a medical device. When formed of different materials, the second magnetic susceptibility, or second susceptibility, can be the same as, or different than, the fourth magnetic susceptibility, or fourth susceptibility, depending on various considerations, such as how a material is applied to a medical device.

For example, using the superposition principle of passive susceptibility-based markers (i.e., the additive or subtractive property of magnetic field distortions due to overlapping markers), the first medical device 10 and the second medical device 50, each with their own respective marker 46, 78, can be used to alter one or more image artifacts generated under MRI when the medical devices 10, 50 are used in combination. For example, image artifacts generated by the second marker 78 under MRI can be attenuated, or completely, or partially, eliminated, by the first marker 46, or vice versa, when the first medical device 10 and the second device 50 are used such that the first marker 46 overlaps (e.g., axially) the second marker 78. In this embodiment, the first marker 46 has an equal but opposing magnetic susceptibility (e.g., opposite sign, opposite magnetic susceptibility) relative to the second marker 78. Alternative embodiments, however, can include a first marker that has a non-equal opposing magnetic susceptibility relative to a second marker. Alternatively, image artifacts generated by the second marker 78 under MRI could be amplified by the first marker 46, or vice versa, when the first medical device 10 and the second medical device 50 are used such that the first marker 46 overlaps the second marker 78. In this embodiment, the first marker 46 has the same magnetic susceptibility (e.g., same sign, an equal magnetic susceptibility, non-equal magnetic susceptibility), or same susceptibility, or a greater magnetic susceptibility, as the second marker 78. When a first and second marker overlap, an altered image artifact is created under MRI that has a third set of characteristics (e.g., size, shape, position, orientation, magnetic susceptibility, intensity) that are different than the first and second set of characteristics.

Use of the medical system 8 and first and second medical devices 10, 50 in this way is considered advantageous at least because the image artifacts created under MRI can be altered dynamically, in real time, during the performance of an MRI-guided procedure or across multiple MRI-guided procedures, as described herein. For example, by changing the relative position of the first and second medical devices 10, 50 in real-time, a dynamic range of image artifacts can be created under MRI. For example, a first marker on a first medical device and a second marker on a second medical device can act independently (e.g., when not overlapping) or collectively (e.g., when overlapping) on an external magnetic field depending on their location relative to one another such that various distinct image artifacts can be created under MRI. Alternatively, a first medical device and a second medical device can be attached to one another, or disposed relative to one another, such that the image artifact created by a marker on one of the first and second medical devices under MRI is statically altered (e.g., by another marker, by the other medical device) during the performance of an MRI-guided procedure, or across multiple MRI-guided procedures.

By leveraging paramagnetic, ferromagnetic, and diamagnetic properties of materials, a clinician is able to amplify, attenuate, or effectively eliminate all, or the majority of, an image artifact near a region of interest. For example, when obtaining a prostate biopsy, little to no image artifact may be desired at, or near, the tip of a cannula (e.g., needle cannula), such as one formed of titanium. Therefore, a first medical device can be formed of titanium and a second medical device, or portion of a second medical device (e.g., distal end portion, marker), can be formed of a material (e.g., polycrystalline graphite) that eliminates, or attenuates, an image artifact produced by the first medical device during use. Pure titanium has a magnetic susceptibility of ˜160 ppm and polycrystalline graphite has a magnetic susceptibility of ˜−200 ppm. Using these materials concentrically would result in near cancelation of any susceptibility effects, resulting in little to no image artifact under MRI.

While the first medical device 10 has been illustrated as including one marker 46 positioned at a particular location on the first medical device 10 and the second medical device 50 has been illustrated as including one marker 78 positioned at a particular location on the second medical device 50, any suitable number of markers can be positioned at any suitable location on a medical device according to an embodiment. Examples of suitable numbers of markers for inclusion in a medical device according to an embodiment include one marker, two markers, more than two markers, three markers, a plurality of markers, four markers, five markers, six markers, seven markers, eight markers, nine markers, ten markers, and more than ten markers. Furthermore, in medical devices that include two or more markers, the markers can be spaced from each other by any desired distance (e.g., equally spaced, varying distances between adjacent markers). It should be noted, though, that, because the markers produce visual artifacts and their utility in the medical device is based on this production of visual artifacts in MRI procedures, it is desirable to space markers disposed on a single medical device from each other by a distance that does not result in the medical device producing overlapping or nearly overlapping visual artifacts. However, alternative embodiments can include a first medical device in which the material forming a portion, or the entirety, of an elongate member of the first medical device can be utilized as a marker, as described herein, to alter an image artifact of a second medical device disposed through the first medical device. Alternatively, alternative embodiments can include a second medical device in which the material forming a portion, or the entirety, of an elongate member of the second medical device can be utilized as a marker, as described herein, to alter an image artifact of a first medical device through which the second medical device can be disposed.

While the elongate member 16 of the cannula 12 has been illustrated as including a distal tapered tip 42 with a cutting edge 44 and the elongate member 56 of the stylet 52 has been illustrated as including a distal tapered tip 74 with a cutting edge 76, an elongate member included on a medical device can include any suitable structural configuration, such as those with blunted, or non-tapered, distal tips. Furthermore, in alternative embodiments a hub member can include any suitable structure to accomplish an attachment to another device, a hub member can be omitted from a medical device, and/or a stylet can be hollow such that it defines a lumen that extends the entire length of the stylet or a portion of the length of the stylet.

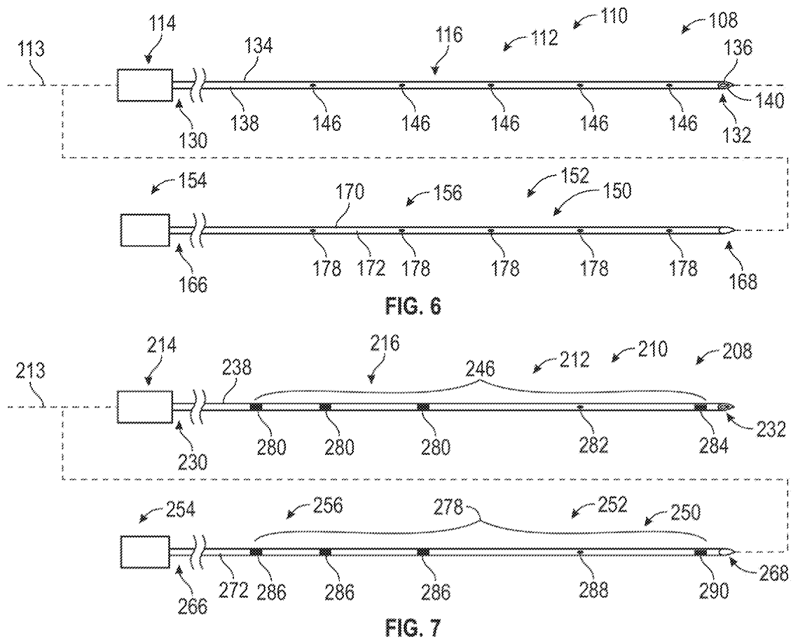

FIG. 6 illustrates a second example medical system 108 useful in performing treatment under MRI (e.g., altering an image artifact under MRI, altering characteristics of an image artifact under MRI). In this example, the second example medical system 108 includes a first medical device 110 and a second medical device 150. In the illustrated embodiment, the first medical device 110 is a cannula 112 and the second medical device 150 is a stylet 152 that can be used coaxially with the first medical device 110. The cannula 112 is similar to the cannula 12 illustrated in FIGS. 1, 2, and 4 and described above, except as detailed below. The stylet 152 is similar to the stylet 52 illustrated in FIGS. 1, 3, and 5 and described above, except as detailed below.

In the illustrated embodiment, the cannula 112 has a lengthwise axis 113, a hub member 114, and an elongate member 116. The elongate member 116 has a proximal end 130, a distal end 132, an outer surface 134, inner surface 136, and a main body 138 that defines a lumen 140. The lumen 140 extends from the proximal end 130 to the distal end 132 such that one or more devices can be passed into, and through, the elongate member 116 of the cannula 112. The elongate member 116 of the cannula 112 is formed of a first material that has a first magnetic susceptibility.

In the embodiment shown, the elongate member 116 of the cannula 112 includes a first plurality of markers 146 disposed between the proximal end 130 and the distal end 132 of the elongate member 116. Each marker of the first plurality of markers 146 is disposed only around a portion of the circumference of the main body 138 of the elongate member 116 of the cannula 112. Each marker in the first plurality of markers 146 is equally spaced from an adjacent marker in the first plurality of markers 146 along the elongate member 116. Each marker in the first plurality of markers 146 is a passive marker and is formed of a second material that is different than the first material. The second material has a second magnetic susceptibility that is different than (e.g., greater than) the first magnetic susceptibility. Each marker in the first plurality of markers 146 creates an image artifact under MRI that has a first set of characteristics (e.g., size, shape, position, orientation, magnetic susceptibility, intensity).

In the illustrated embodiment, the stylet 152 has a hub member 154 and an elongate member 156. The elongate member 156 has a proximal end 166, a distal end 168, an outer surface 170, and a main body 172 that defines a solid member. The elongate member 156 of the stylet 152 can be passed into, and through, the lumen 140 of the elongate member 116 of the cannula 112. The elongate member 156 of the stylet 152 is formed of a third material that has a third magnetic susceptibility. In the illustrated embodiment, the first material that forms the elongate member 116 of the cannula 112 and the third material that forms the elongate member 156 of the stylet 152 are different (e.g., have different magnetic susceptibilities).

In the embodiment illustrated, the elongate member 156 of the stylet 152 includes a second plurality of markers 178 disposed between the proximal end 166 and the distal end 168. Each marker of the second plurality of markers 178 is disposed only around a portion of the circumference of the main body 172 of the elongate member 156 of the stylet 152. Each marker in the second plurality of markers 178 is equally spaced from an adjacent marker in the second plurality of markers 178 along the elongate member 156. Each marker in the second plurality of markers 178 is a passive marker and is formed of a fourth material that is different than the first material and the third material and that has a fourth magnetic susceptibility that is different than (e.g., greater than) the first magnetic susceptibility and the third magnetically susceptibility. Each marker in the second plurality of markers 178 creates an image artifact under MRI that has a second set of characteristics (e.g., size, shape, position, orientation, magnetic susceptibility, intensity) that are different than the first set of characteristics. Each marker in the second plurality of markers 178 is positioned on the elongate member 156 such that each marker in the first plurality of markers 146 can overlap (e.g., axially) a marker in the second plurality of markers 178 when the second medical device 150 is disposed within the first medical device 110 (e.g., each marker in the first plurality of markers 146 is disposed at the same location along the lengthwise axis 113 of the elongate member 116 of the cannula 112 as a marker in the second plurality of markers 146).

In the illustrated embodiment, each marker in the first plurality of markers 146 and each marker in the second plurality of markers 178 is a deposit of material attached to their respective elongate members 116, 156 and is formed as a dot. The first material that forms the elongate member 116 of the first medical device 110, the second material that forms the elongate member 156 of the second medical device 150, and the material that forms a marker included on a medical device can comprise any suitable material having the magnetic susceptibility described herein. For example, the first material, the second material, the third material, and the fourth material can comprise any suitable MRI compatible material having the magnetic susceptibility described herein. In the embodiment illustrated, the first material comprises titanium, the second material comprises a polymer, and each marker in the first plurality markers 146 is formed of second material that has a second magnetic susceptibility that alters an image artifact created by each marker in the second plurality markers 178 under MRI when a marker of the first plurality of markers 146 overlaps a marker of the second plurality of markers 178. When the first plurality of markers 146 and the second plurality of markers overlap, a plurality of altered image artifacts is created under MRI that has a third set of characteristics (e.g., size, shape, position, orientation, magnetic susceptibility, intensity) that are different than the first and second set of characteristics. Alternatively, each marker in a second plurality markers can be formed of a fourth material that has a fourth magnetic susceptibility that alters an image artifact created by each marker in a first plurality markers under MRI when a marker of the first plurality of markers overlaps a marker of the second plurality of markers during use. In the embodiment shown, this is accomplished by forming each marker in the first plurality of markers 146 of a material that is different than the material that forms each marker in the second plurality of markers 178 such that the second material has a second magnetic susceptibility that is different than the fourth magnetic susceptibility (e.g., the magnetic susceptibilities have different signs). In this embodiment, image artifacts generated by the markers in the second plurality of markers 178 under MRI are attenuated by the markers in the first plurality of markers 146, or vice versa, when the first medical device 110 and the second device 150 are used such that the first plurality of markers 146 overlaps the second plurality of markers 178. Alternative embodiments can include a first plurality of markers formed of a second material that has a second magnetic susceptibility, or a second susceptibility, that is the same as, or different than, a fourth magnetic susceptibility, or fourth susceptibility, of a fourth material that forms a second plurality of markers such that the magnetic susceptibility, or susceptibility, of the second material and/or fourth material is amplified (e.g., the magnetic susceptibilities have the same signs), attenuated, or eliminated, and the resulting image artifact under MRI is amplified, attenuated, or eliminated.

Use of the medical system 108 and the first and second medical devices 110, 150 is considered advantageous at least because the image artifacts created by the first plurality of markers 146 and/or second plurality of markers 178 can be altered dynamically, in real time, during the performance of an MRI-guided procedure or across multiple MRI-guided procedures, as described herein. For example, by changing the relative position of the first and second medical devices 110, 150 in real-time, a dynamic range of image artifacts created under MRI can be created.

FIG. 7 illustrates a third example medical system 208 useful in performing treatment under MRI (e.g., altering an image artifact under MRI). In this example, the second example medical system 208 includes a first medical device 210 and a second medical device 250. In the illustrated embodiment, the first medical device 210 is a cannula 212 and the second medical device 250 is a stylet 252 that can be used coaxially with the first medical device 210. The cannula 212 is similar to the cannula 112 illustrated in FIG. 6 and described above, except as detailed below. The stylet 252 is similar to the stylet 152 illustrated in FIG. 6 and described above, except as detailed below.

In the illustrated embodiment, the cannula 212 has a lengthwise axis 213, a hub member 214, and an elongate member 216. The elongate member 216 of the cannula 212 is formed of a first material that has a first magnetic susceptibility. In the embodiment shown, the elongate member 216 of the cannula 212 includes a first plurality of markers 246 disposed between the proximal end 230 and the distal end 232 of the elongate member 216. Each marker in the first plurality of markers 246 is a passive marker. The first plurality of markers 246 are spaced along the elongate member 216 at varying distances from one another. The first plurality of markers 246 includes a first subset of markers 280, a second subset of markers 282, and a third subset of markers 284. Each marker in the first subset of markers 280 is disposed between the proximal end 230 and the distal end 232 of the elongate member 216 of the first medical device 210, around the entire circumference of the main body 238 of the elongate member 216, and is formed of a second material that is different than the first material. The second material has a second magnetic susceptibility that is different than (e.g., greater than) the first magnetic susceptibility. Each marker in the first subset of markers 280 creates an image artifact under MRI that has a first set of characteristics (e.g., size, shape, position, orientation, magnetic susceptibility, intensity). Each marker in the second subset of markers 282 is disposed between the first subset of markers 280 and the third subset of markers 284, is disposed only around a portion of the circumference of the main body 238, and is formed of a third material that is different than the first material and the second material. The third material has a third magnetic susceptibility that is different than (e.g., greater than) the first magnetic susceptibility and different than (e.g., greater than, less than) the second magnetic susceptibility. Each marker in the second subset of markers 282 creates an image artifact under MRI that has a second set of characteristics (e.g., size, shape, position, orientation, magnetic susceptibility, intensity) that are different than the first set of characteristics. Each marker in the third subset of markers 284 is disposed between the second subset of markers 282 and the distal end 232 of the elongate member 216, is disposed only around a portion of the circumference of the main body 238, and is formed of a fourth material that is different than the first material, the second material, and the third material. The fourth material has a fourth magnetic susceptibility that is different than (e.g., greater than) the first magnetic susceptibility, different than (e.g., greater than, less than) the second magnetic susceptibility, and different than (e.g., greater than, less than) the third magnetic susceptibility. Each marker in the third subset of markers 284 creates an image artifact under MRI that has a third set of characteristics (e.g., size, shape, position, orientation, magnetic susceptibility, intensity) that are different than the first set of characteristics and the second set of characteristics. The first subset of markers 280 includes three markers, the second subset of markers 282 includes one marker, and the third subset of markers 284 includes one marker. However, alternative embodiments can include any suitable number and type of markers in a subset of a plurality of markers and each subset of markers can be formed of any suitable material having the magnetic susceptibility described herein.

In the embodiment shown, each marker in the first subset of markers 280 is a band of material attached to the elongate member 216, each marker in the second subset of markers 282 is a deposit of material attached to the elongate member 216 and is formed as a dot, and each marker in the third subset of markers 284 is a deposit of material attached to the elongate member 216 and is formed as a rectangle. While the markers in the first plurality of markers 246 have been shown as being disposed at particular locations on the elongate member 216 and as having a particular structural arrangement, a marker included on a medical device can be positioned at any suitable location and have any suitable structural arrangement.

In the illustrated embodiment, the stylet 252 has a hub member 254 and an elongate member 256. The elongate member 256 of the stylet 252 is formed of a fifth material that has a fifth magnetic susceptibility. In the illustrated embodiment, the first material that forms the elongate member 216 of the cannula 212 and the fifth material that forms the elongate member 256 of the stylet 252 are different.

In the embodiment shown, the elongate member 256 of the stylet 252 includes a second plurality of markers 278 disposed between the proximal end 266 and the distal end 268 of the elongate member 256. Each marker in the second plurality of markers 278 is a passive marker. The second plurality of markers 278 are spaced along the elongate member 256 at varying distances from one another. The second plurality of markers 278 includes a first subset of markers 286, a second subset of markers 288, and a third subset of markers 290. Each marker in the first subset of markers 286 is disposed between the proximal end 266 and the distal end 268 of the elongate member 256 of the second medical device 250, around the entire circumference of the main body 272 of the elongate member 256, and is formed of a sixth material that is different than the fifth material. The sixth material has a sixth magnetic susceptibility that is different than (e.g., greater than) the fifth magnetic susceptibility. Each marker in the first subset of markers 286 creates an image artifact under MRI that has a first set of characteristics (e.g., size, shape, position, orientation, magnetic susceptibility, intensity). Each marker in the second subset of markers 288 is disposed between the first subset of markers 286 and the third subset of markers 290, is disposed only around a portion of the circumference of the main body 272, and is formed of a seventh material that is different than the fifth material and the sixth material. The seventh material has a seventh magnetic susceptibility that is different than (e.g., greater than) the fifth magnetic susceptibility and different than (e.g., greater than, less than) the sixth magnetic susceptibility. Each marker in the second subset of markers 288 creates an image artifact under MRI that has a second set of characteristics (e.g., size, shape, position, orientation, magnetic susceptibility, intensity) that are different than the first set of characteristics. Each marker in the third subset of markers 290 is disposed between the second subset of markers 288 and the distal end 268 of the elongate member 256 of the second medical device 250, is disposed only around a portion of the circumference of the main body 272, and is formed of an eighth material that is different than the fifth material, the sixth material, and the seventh material. The eighth material has an eighth magnetic susceptibility that is different than (e.g., greater than) the fifth magnetic susceptibility, different than (e.g., greater than, less than) the sixth magnetic susceptibility, and different than (e.g., greater than, less than) the seventh magnetic susceptibility. Each marker in the third subset of markers 290 creates an image artifact under MRI that has a third set of characteristics (e.g., size, shape, position, orientation, magnetic susceptibility, intensity) that are different than the first set of characteristics and the second set of characteristics. The first subset of markers 286 includes three markers, the second subset of markers 288 includes one marker, and the third subset of markers 290 includes one marker. However, alternative embodiments can include any suitable number and type of markers in a subset of a plurality of markers and each subset of markers can be formed of any suitable material having the magnetic susceptibility described herein.

In the embodiment shown, each marker in the first subset of markers 286 is a band of material attached to the elongate member 256, each marker in the second subset of markers 288 is a deposit of material attached to the elongate member 256 and is formed as a dot, and each marker in the third subset of markers 290 is a deposit of material attached to the elongate member 256 and is formed as a rectangle. While the markers in the second plurality of markers 278 have been shown as being disposed at particular locations on the elongate member 256 and as having a particular structural arrangement, a marker included on a medical device can be positioned at any suitable location and have any suitable structural arrangement.

Each marker in the second plurality of markers 278 is positioned on the elongate member 256 such that the first plurality of markers 246 overlaps (e.g., axially) the second plurality of markers 279 when the second medical device 250 is disposed within the first medical device 210 and the plurality of markers 246, 278 are aligned relative to the lengthwise axis 213 of the elongate member 216 of the cannula 212. For example, each marker in the second plurality of markers 278 is positioned on the elongate member 256 such that a marker in the first plurality of markers 246 can overlap a marker in the second plurality of markers 278 when the second medical device 250 is disposed within the first medical device 210 and the marker in the first plurality of markers 246 and the marker in the second plurality of markers 278 is disposed at the same location along the lengthwise axis 213 of the elongate member 216 of the cannula 212.

The first material that forms the elongate member 216 of the first medical device 210, the second material that forms the elongate member 256 of the second medical device 250, and the material that forms a marker included on a medical device can comprise any suitable material. For example, the first material, the second material, the third material, the fourth material, the fifth material, the sixth material, the seventh material, and the eighth material can comprise any suitable MRI compatible material having the magnetic susceptibility described herein. In the embodiment illustrated, the first material comprises titanium, the second material comprises a polymer, and each marker in the first plurality markers 246 is formed of a material that has a magnetic susceptibility that alters an image artifact of each marker in the second plurality markers 278 under MRI when a marker of the first plurality of markers 246 overlaps (e.g., axially) a marker of the second plurality of markers 278 during use. Alternatively, each marker in a second plurality markers can be formed of a material that has a magnetic susceptibility that alters an image artifact created by each marker in a first plurality markers under MRI when a marker of the first plurality of markers overlaps (e.g., axially) a marker of the second plurality of markers during use.

Each marker in the first subset of markers 280 is formed of a material that is different than the material that forms each marker in the first subset of markers 286 such that the second material has a second magnetic susceptibility that is different (e.g., greater than, less than) than the sixth magnetic susceptibility (e.g., the magnetic susceptibilities have different signs). Each marker in the second subset of markers 282 is formed of a material that is different than the material that forms each marker in the second subset of markers 288 such that the third material has a third magnetic susceptibility that is different than (e.g., greater than, less than) the seventh magnetic susceptibility (e.g., the magnetic susceptibilities have different signs). Each marker in the third subset of markers 284 is formed of a material that is different than the material that forms each marker in the third subset of markers 290 such that the fourth material has a fourth magnetic susceptibility that is different than (e.g., greater than, less than) the eighth magnetic susceptibility (e.g., the magnetic susceptibilities have different signs).

In the embodiment illustrated, when the first medical device 210 and the second device 250 are used such that the first plurality of markers 246 overlaps (e.g., axially) the second plurality of markers 278, image artifacts generated by the markers in the first subset of markers 286 under MRI are amplified by the markers in the first subset of markers 280, or vice versa, image artifacts generated by the markers in the second subset of markers 288 under MRI are amplified by the markers in the second subset of markers 282, or vice versa, and image artifacts generated by the markers in the third subset of markers 290 under MRI are amplified by the markers in the third subset of markers 284, or vice versa. When a marker in the first subset of markers 280 and a marker in the first subset of markers 286 overlap, an altered image artifact is created under MRI that has a fourth set of characteristics (e.g., size, shape, position, orientation, magnetic susceptibility, intensity) that are different than the characteristics of the markers included in the first subset of markers 280 and the first subset of markers 286. When a marker in the second subset of markers 282 and a marker in the first subset of markers 288 overlap, an altered image artifact is created under MRI that has a fifth set of characteristics (e.g., size, shape, position, orientation, magnetic susceptibility, intensity) that are different than the characteristics of the markers included in the second subset of markers 282 and the second subset of markers 288. When a marker in the third subset of markers 284 and a marker in the third subset of markers 288 overlap, an altered image artifact is created under MRI that has a sixth set of characteristics (e.g., size, shape, position, orientation, magnetic susceptibility, intensity) that are different than the characteristics of the markers included in the third subset of markers 284 and the third subset of markers 290. Alternative embodiments can include a subset of markers on a first medical device that are formed of the same material as a corresponding subset of markers on a second medical device that have the same magnetic susceptibility, or same susceptibility, such that image artifacts created by the markers in each subset under MRI is attenuated, eliminated, or amplified (e.g., the magnetic susceptibilities have the same signs).

Furthermore, depending on the position of the first and second medical devices 210, 250 relative to one another, the markers of the third subset of markers 284 can alter image artifacts generated by the markers in the second subset of markers 288 under MRI or the image artifacts generated by the markers in the first subset of markers 286 under MRI, or vice versa. In addition, depending on the position of the first and second medical devices 210, 250 relative to one another, the markers of the second subset of markers 282 can alter image artifacts generated by the markers in the first subset of markers 286 under MRI, or vice versa.

Use of the medical system 208 and the first and second medical devices 210, 250 is considered advantageous at least because image artifacts created by a marker included in one of the first and second plurality of markers 246, 278 under MRI can be altered dynamically, in real time, during the performance of an MRI-guided procedure or across multiple MRI-guided procedures, as described herein. For example, by changing the relative position of the first and second medical devices 210, 250 in real-time, a dynamic range of image artifacts can be created under MRI.

While the markers shown in the medical devices described herein have been shown as a band of material, a material deposited on a medical device and forming a dot, and/or a material deposited on a medical device and forming a rectangle, a marker can comprise any suitable structure attached to a medical device (e.g., stylet, cannula) or any suitable treatment imparted on a medical device. For example, a marker can include bands of material, magnetic inks, sputtered magnetite, dimpling, swagging, or peening the material that forms a portion of a medical device (e.g., annealed 304 stainless steel), a single layer or multiple layers of a material (e.g., metal, metal alloy), or materials (e.g., metals, metal alloys), deposited along an outer surface and/or an inner surface of a medical device (e.g., elongate member of a medical device), and/or any other material considered suitable to form a distinct marker (e.g., image artifact) visible under MRI and having the magnetic susceptibility described herein. Any suitable marker having the magnetic susceptibility described herein can be included in a medical device. Examples of markers considered suitable to include in a medical device are described in U.S. patent application Ser. No. 16/454,905, filed on Jun. 27, 2019, which is hereby incorporated by reference in its entirety for the purpose of describing markers considered suitable to include in a medical device.

Each marker included on a medical device can have any suitable structural arrangement and characteristic under MRI. For example, a marker can comprise a structure that defines any suitable shape, such as a curve along a portion of a length of an elongate member, a geometric shape on a portion of an elongate member (e.g., circle, polygon), a spiral along a portion of a length of an elongate member, any combination of shapes described herein, or any other shape considered suitable for a particular embodiment. The configurations described herein provide a mechanism for creating a marker that has a resulting image artifact created by the marker under MRI that is distinguishable from the surrounding fluid and/or tissue being imaged under MRI.

While the medical devices described herein have included cannulas and stylets, the markers described herein can be included on any suitable medical devices that can be used in combination with one another (e.g., coaxially) and selection of a suitable medical device to include a marker can be based on various considerations including the intended use of the medical device on which a marker is disposed. Examples of medical devices considered suitable to include a marker include cannulas, needles, stylets, sheaths, catheters, balloon catheters, dilators, guidewires, introducers, implantable devices, two medical devices that can be used in combination with one another (e.g., coaxially), such as a needle and guidewire, a guidewire and an introducer, a guidewire and a catheter, a guidewire and a sheath, a sheath and a catheter, an implantable medical device and an associated delivery system, any medical device capable of being passed over, or within, a guidewire, and any other medical device considered suitable for a particular embodiment. Furthermore, the features described herein can be utilized on more than two medical devices, that can be used independently, or in combination with one another.

A first medical device can comprise any suitable type of medical device. For example, a first medical device can comprise any suitable type of cannula having any suitable length and gauge. For example, a first medical device can comprise an Inconel Chiba Needle, an Inconel micropuncture needle, or any other needle considered suitable for a particular embodiment, have a length equal to, greater than, or less than 7 centimeters, 15 centimeters, or any other length considered suitable for a particular embodiment, and/or have a gauge equal to, greater than, or less than 20, 21, 22, or any other gauge considered suitable for a particular embodiment. A second medical device can comprise any suitable type of medical device. For example, a second medical device can comprise any suitable type of stylet having any suitable length and gauge.

In addition, the medical devices described herein can be used with any suitable number of outer sheaths to accomplish treatment. Selection of a suitable number of outer sheaths to utilize when completing sequential dilation, for example, can be based on the bodily passage within which a sequential dilation is being completed. Examples of numbers of outer sheaths considered suitable to utilize when completing sequential dilation include one, two, a plurality, three, four, more than four, and any other number considered suitable for a particular embodiment.

While the marker(s) on a first medical device have been described as overlapping the marker(s) on a second medical device such that an altered image artifact is created in an MR image, such overlapping can include a partial overlap between two markers (e.g., two coaxial makers) or a complete overlap between two markers (e.g., two coaxial makers) and will be dependent upon the structural arrangement (e.g., size, shape, position, magnetic susceptibility, intensity) of the markers intended to overlap one another.

Any type of alteration of an image artifact can be accomplished by overlapping two markers and will be dependent upon the size, shape, position, magnetic susceptibility, and/or the material that forms the markers. Examples of types of alterations that can be accomplished using overlapping markers include altering a size, a shape, an intensity, and/or an appearance of an image artifact (e.g., eliminate, amplify, attenuate), and any other alteration considered suitable for a particular embodiment.

While particular materials have been described as forming a first medical device (e.g., elongate member), a second medical device (e.g., elongate member), and marker(s), a medical device, or portion thereof (e.g., elongate member), and a marker can be formed of any suitable material having the magnetic susceptibility described herein to accomplish the features described herein. For example, a first medical device, a second medical device, or portions thereof (e.g., elongate member), and a marker, or markers, included on a medical device can be formed of any suitable MRI compatible material having the magnetic susceptibility described herein and selection of a suitable material can be based on various considerations, including the intended use of the first medical device, the second medical device, and/or the marker(s). In addition, or in an alternative embodiment, a first medical device, a second medical device, or portions thereof (e.g., elongate member), a marker, or markers, included on a medical device, and/or any other material described herein can comprise any suitable MRI compatible material having a susceptibility, or relative susceptibility, similar to the magnetic susceptibility, or relative magnetic susceptibilities, described herein relative to the portion of a system or device which the material forms. Examples of materials considered suitable to form a first medical device, a second medical device, and/or a marker, or markers, or portion thereof, include biocompatible materials, materials that can be made biocompatible, MRI compatible materials, metals, electrically insulating materials, electrically non-conducting materials, non-ferromagnetic materials, such as a non-magnetically susceptible materials, non-diamagnetic materials, passive materials, magnetically susceptible materials, including diamagnetic materials (e.g., pyrolytic carbon), paramagnetic materials, or ferromagnetic materials, ferromagnetic passive materials, Ferritic Stainless Steel, Ferritic Stainless Steel 430L powder, shape memory alloys, including nickel-titanium alloys, such as Nitinol, austentic nickel-chromium based alloys, such as Inconel, a brand for a family of austenitic nickel-chromium-based superalloys from Special Metals Corporation, stainless steel, 304V SS, including Austenitic stainless steel, stainless steel containing Iron, stainless steel including Inconel, iron, cobalt, cobalt chromium, cobalt chromium alloys, titanium, materials (e.g., stainless steel) having a hardness of about 192 KSI, nickel, nickel plating, bright nickel plating, alloys that include less than 1% of iron by weight, metals that include murinite, such as MuMetal, which is a brand for a nickel-iron ferromagnetic alloy with a very high permeability from Magnetic Shield Corporation, Gadolinium, pyrolytic carbon, silver, graphite, polycrystalline graphite, plastics, polymers, PEEK, carbon-filled PEEK, polyethylene, such as high-density polyethylene (HDPE), polypropylene, polycarbonates, silicone, Delrin, ceramics, transparent materials, opaque materials, ceramics, the materials described herein, combinations of the described herein, and any other material considered suitable for a particular embodiment and having the magnetic susceptibility described herein. Overlapping markers, or markers that can be overlapped during performance of a procedure, can be formed of any suitable materials, such as those described herein, paramagnetic materials (e.g., used to amplify an image artifact, materials that contain iron, nickel, cobalt (e.g., 300 series stainless steel), nickel-cobalt, cobalt chromium), ferromagnetic materials (e.g., used to amplify an image artifact, materials that contain iron, nickel, cobalt (e.g., 300 series stainless steel), nickel-cobalt, cobalt chromium), diamagnetic materials (e.g., used to attenuate or eliminate an image artifact, pyrolytic carbon, silver, carbon (e.g., graphite)), and any other material considered suitable for a particular embodiment.

Selection of a marker, or markers, to include on a medical device can also be based upon the field strength, or field strengths, within which the medical device is intended to be used. For example, a medical device that includes a marker can be utilized to complete one, or more than one, MRI-guided interventional procedure utilizing one or more field strengths (0.55 Tesla (T), 1.5 T, or 3.0 T). Cumulative susceptibility-based artifacts that result from overlapping two or more markers can be selected to respond differently to a single imaging sequence, or multiple imaging sequences (gradient echo vs. spin echo), or at different magnetic field strengths. As described herein, the medical devices are not limited to being used to alter the size, shape, and intensity of a single image artifact, and instead a series of susceptibility markers across multiple devices can be combined (statically or dynamically) to change the geometry of a larger (or smaller) cumulative artifact, which can lead to the creation of distinct image artifacts that are easily identifiable under MRI.

As described herein, visualization of devices during the performance of a procedure is a fundamental challenge in interventional MRI. Conventional interventional devices and materials are either highly susceptible, resulting in artifacts that obliterate key features of the imaged tissue/field, or minimally susceptible, leaving devices not visible for identification or tracking within the image. Therefore, there is a need to leverage diagnostic imaging modalities, MR in particular, directly in guiding therapy. Identification and precise targeting of lesions or abnormal anatomy is a significant clinical challenge. The medical devices described herein are considered advantageous at least because they can substantially improve the ability of clinicians to precisely visualize, identify, track, and navigate medical devices to various locations within the body and improve overall patient care (e.g., diagnosis, treatment delivery). The medical devices described herein provide markers that can be used to generate identifiable, manipulatable, image artifacts (e.g., patterns) on interventional MR images allowing for a high degree of control over the position, size, and intensity of a marker and associated medical device under MRI.

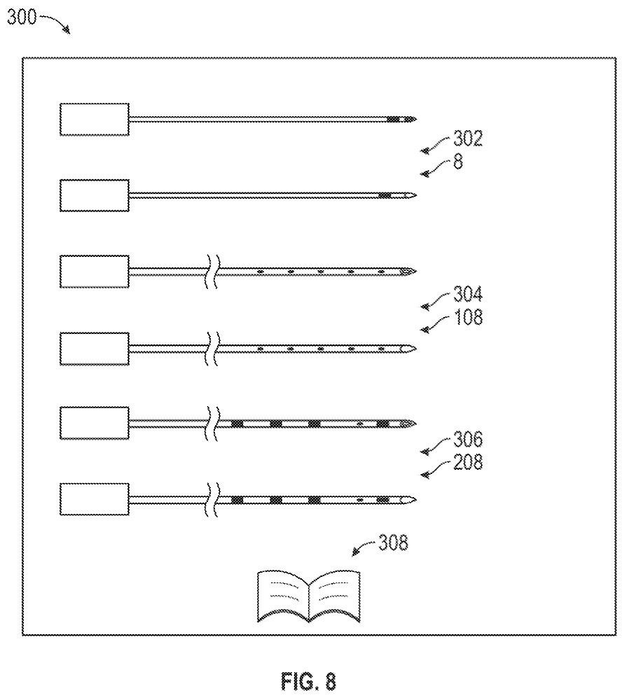

FIG. 8 illustrates an example kit 300 useful for performing treatment under MRI. The kit 300 includes a first medical system 302 according to an embodiment, such as the first medical system 8 illustrated in FIGS. 1, 2, 3, 4, and 5; a second medical system 304 according to an embodiment, such as the second medical system 108 illustrated in FIG. 6; a third medical system 306 according to an embodiment, such as the third medical system 208 illustrated in FIG. 7; and instructions for use 308.

While kit 300 has been illustrated as including three medical systems 302, 304, and 306, any suitable number, and type, of medical systems and/or medical devices can be included in a kit. Selection of a suitable number of medical systems and/or medical devices to include in a kit according to a particular embodiment can be based on various considerations, such as the treatment intended to be performed. Examples of numbers of medical systems and/or medical devices considered suitable to include in a kit include at least one, one, two, a plurality, three, four, five, six, seven, eight, nine, ten, more than ten, and any other number considered suitable for a particular embodiment.

Furthermore, while medical system 8, medical system 108, and medical system 208 have been illustrated as included in kit 300, any suitable medical system and/or medical device can be included in a kit. Selection of a suitable medical system and/or medical device to include in a kit according to a particular embodiment can be based on various considerations, such as the treatment intended to be performed. Examples of medical systems and medical devices considered suitable to include in a kit include those described herein, variations of those described herein, one or more inner sheaths, one or more outer sheaths, one or more access needles, one or more implantable medical devices, one or more delivery systems, and/or any other medical device according to an embodiment considered suitable for a particular embodiment.

For example, in embodiments in which a kit includes a plurality of medical devices (e.g., a needle and a plurality of stylets), a first medical device (e.g., stylet) can include a marker formed of a material that forms an image artifact under a first MR field strength (e.g., 0.55 T), a second medical device (e.g., stylet) can include a marker formed of a material that forms an image artifact under a second MR field strength (e.g., 1.5 T), and a third medical device (e.g., stylet) can include a marker formed of a material that forms an image artifact under a third MR field strength (e.g., 3 T). The first MR field strength can be different, or the same as, the second MR field strength and/or the third MR field strength. The second MR field strength can be different, or the same as, the first MR field strength and/or the third MR field strength. The third MR field strength can be different, or the same as, the first MR field strength and/or the second MR field strength. Each of the first, second, and third medical devices (e.g., stylets) can be used with a fourth medical device (e.g., needle) to perform a procedure.

In an example, a kit useful in performing treatment under magnetic resonance includes a first medical device of an embodiment, a second medical device of an embodiment, and a third medical device of an embodiment. The first medical device includes a first marker that produces a first image artifact under a first MR field strength. The second medical device includes a second marker that produces a second image artifact under a second MR field strength. The third medical device includes a third marker that produces a third image artifact under a third MR field strength. The first MR field strength is different than the second MR field strength and the third MR field strength. The second MR field strength is different than the third MR field strength. The first image artifact has a first set of characteristics (e.g., size, shape, pattern, and/or intensity) under the first MR field strength, a second set of characteristics under the second MR field strength, and a third set of characteristics under the third MR field strength. The first set of characteristics of the first image artifact being different than the second set of characteristics of the first image artifact and the third set of characteristics of the first image artifact. The second set of characteristics of the first image artifact being different than the third set of characteristics of the first image artifact. The second image artifact has a first set of characteristics under the first MR field strength, a second set of characteristics under the second MR field strength, and a third set of characteristics under the third MR field strength. The first set of characteristics of the second image artifact being different than the second set of characteristics of the second image artifact and the third set of characteristics of the second image artifact. The second set of characteristics of the second image artifact being different than the third set of characteristics of the second image artifact. The third image artifact has a first set of characteristics under the first MR field strength, a second set of characteristics under the second MR field strength, and a third set of characteristics under the third MR field strength. The first set of characteristics of the third image artifact being different than the second set of characteristics of the third image artifact and the third set of characteristics of the third image artifact. The second set of characteristics of the third image artifact being different than the third set of characteristics of the third image artifact.

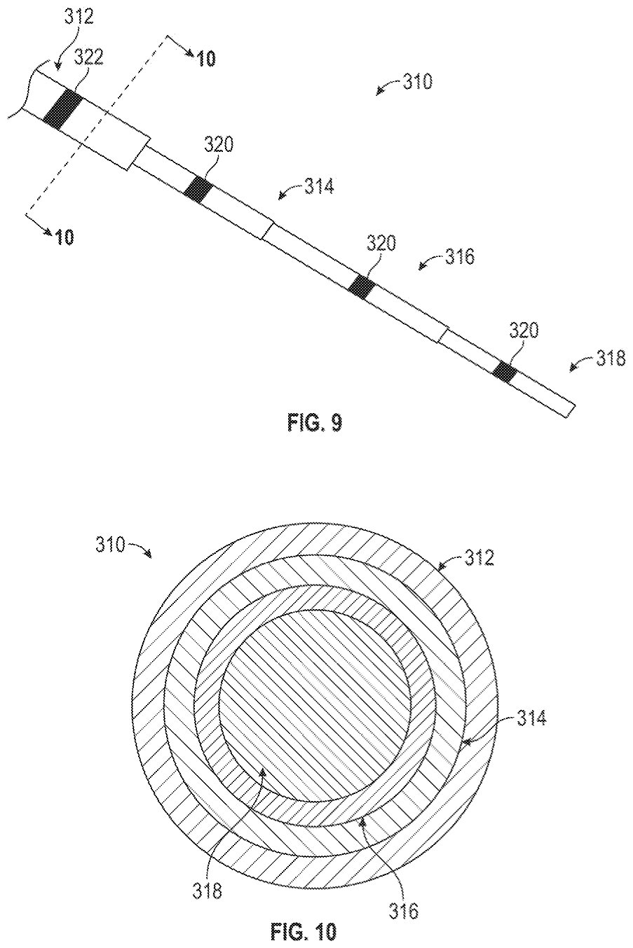

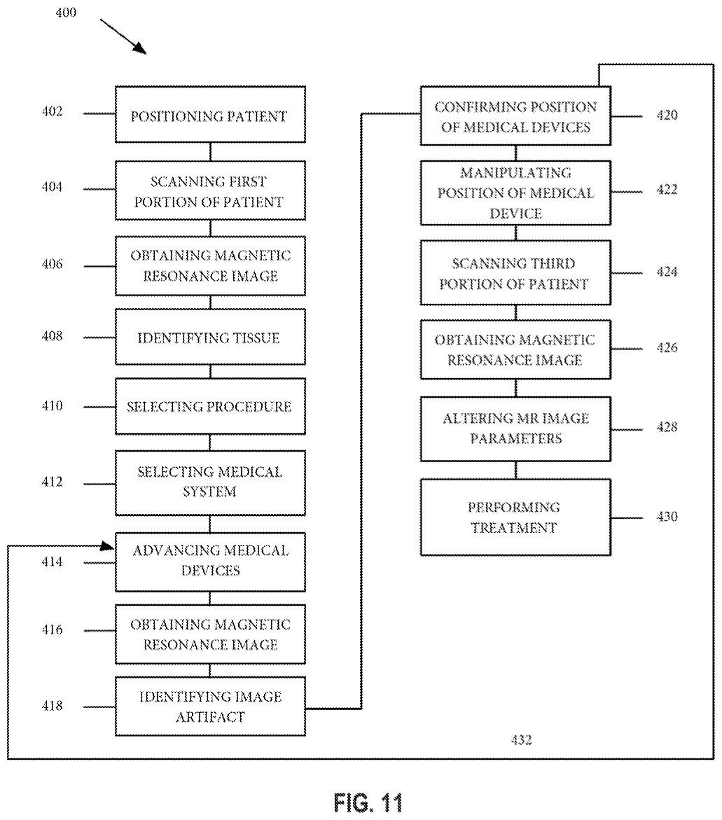

Ina specific embodiment, as shown in FIGS. 9 and 10, a medical system 310 can comprise a first medical device 312, a second medical device 314, a third medical device 316, and a fourth medical device 318 that can be used concentrically. The first medical device 312 comprises a needle (e.g., formed of titanium) according to an embodiment. Each of the second medical device 314, the third medical device 316, and the fourth medical device 318 comprises a stylet according to an embodiment. The first medical device 312 can be packaged with the plurality of stylets 314, 316, 318, each of which can be used in combination with the needle, independently or collectively, depending on a MRI static field strength intended to be used or the specific interventional procedure to be completed. For example, each stylet of the plurality of stylets 314, 316, 318 includes a marker 320 that has a magnetic susceptibility that amplifies, eliminates, or attenuates the magnetic susceptibility created by a marker 322 disposed on the needle 312 under MRI, or vice versa, thereby altering the image artifact of the marker 322 disposed on the needle 312. This can be accomplished in discrete increments (e.g., 0.25×, 0.5×, 2.0×, 4.0×, etc.) depending on the number of markers that overlap. These stylets could then be used independently or collectively to tune the image artifact size in real-time throughout or across multiple procedures. In embodiments in which stylets are used collectively, the stylets can be fabricated using a series of concentric tubes, as illustrated, and any suitable number of concentric tubes can be utilized.

The kits described herein advantageously provide a clinician with a single product (e.g., kit) that could be used under multiple settings. For example, a titanium needle with a low magnetic susceptibility allows for positioning precision near the tip where treatment is being performed (e.g., a biopsy is being taken), meanwhile the markers on one or more of the removable stylets allow for real-time trackability at all field strength or across multiple interventional procedures. While this embodiment describes use of a needle and a plurality of stylets, such an embodiment can include any suitable combination of medical devices, such as those described herein (e.g., sheaths, removable dilators). For example, a sheath formed of a material that creates no, or a slightly-visible, image artifact under MRI can be packaged with a plurality of removable dilators, each of which include one or more markers that create a highly-visible image artifact under MRI to facilitate use under various MRI static field strengths.

Various methods of performing interventional medical treatment under MRI are described herein. While the methods described herein are shown and described as a series of acts, it is to be understood and appreciated that the methods are not limited by the order of acts, as some acts may in accordance with these methods may be omitted, occur in the order shown and/or described, occur in different orders, and/or occur prior to, subsequent to, or concurrently with other acts described herein.

FIG. 11 illustrates a schematic illustration of an example method 400 of performing treatment (e.g., altering an image artifact) under MRI.

An initial step 402 comprises positioning a patient within a MR scanner. Another step 404 comprises scanning a first portion of the patient using the MR scanner. The MR scanner having a first set of MR image parameters and/or sequence parameters. Another step 406 comprises obtaining a MR image of the first portion of the patient. Another step 408 comprises identifying a tissue that has predefined characteristics using the MR image. Another step 410 comprises selecting a procedure to treat the tissue based upon the predefined characteristics. Another step 412 comprises selecting a medical system to accomplish performance of the procedure. The medical system including a first medical device and a second medical device. The first medical device having a first marker and the second medical device having a second marker. While the patient remains positioned within the MR scanner used to scan the first portion of the patient, another step 414 comprises advancing the first medical device and the second medical device (e.g., concentrically) into a bodily passage and to the tissue while scanning a second portion of the patient that includes the first and second medical devices and a first image artifact using the MR scanner. The first image artifact having a first set of characteristics. Another step 416 comprises obtaining a MR image of the second portion of the patient that includes the first and second medical devices and the first image artifact. Another step 418 comprises identifying the first image artifact within the MR image. Another step 420 comprises confirming the position of the first medical device and the second medical device within the bodily passage. Another step 422 comprises manipulating the position of the first medical device relative to the second medical device, or vice versa, to alter the first image artifact to create an altered image artifact. Another step 424 comprises scanning a third portion of the patient that includes the first and second medical devices and the altered image artifact using the MR scanner. Another step 426 comprises obtaining a MR image of the third portion of the patient that includes the first and second medical devices and the altered image artifact. Another step 428 comprises altering (e.g., optimizing) the first set of MR image parameters and/or sequence parameters to a second set of MR image parameters and/or sequence parameters based upon the altered image artifact. Another step 430 comprises performing treatment.

Step 402 can be accomplished by positioning a patient within any suitable MR scanner, having any suitable field strength, such as conventional MR scanners, MR scanners that can accomplish gradient refocusing echo imaging, MR scanners that can accomplish spin echo imaging, MR scanners that can accomplish true fast imaging with steady-state precession, MR scanners that can accomplish fast low flip angle shot spoiled gradient-echo imaging, MR scanners that utilize 0.55 T fields, 1.5 T fields, 3 T fields, fields between about 0.055 T and 1.5 T, fields less than 1 T, and any other MR scanner considered suitable for a particular embodiment.

Step 404 can be accomplished by scanning any suitable portion of a patient and selection of a suitable portion of a patient to scan can be based on various considerations, including the treatment intended to be performed. Examples of portions of a patient considered suitable to scan include the extremities (e.g., arms, legs), chest, breast, spine, neck, head, abdomen, pelvis, prostate, peri-prostatic structures, tissues surrounding the portions of a patient described herein, and/or any other portion of the patient considered suitable for a particular embodiment. An MR scanner can have any suitable number and type of MR image parameters, such as gradient refocusing echo imaging, spin echo imaging, true fast imaging with steady-state precession, fast low flip angle shot spoiled gradient-echo imaging, field strengths, such as 0.55 T, 1.5 T, 3 T, between about 0.055 T and 1.5 T, and fields less than 1 T, slice thickness, flip angle, field-of-view, resolution, gradient fields, and any other image parameter considered suitable for a particular embodiment.

Step 406 can be accomplished by obtaining the MR image from the MR scanner used in step 404.

Step 408 can be accomplished by reviewing the MR image obtained in step 406 and utilizing conventional techniques and/or methods to determine whether tissue has predefined characteristics (e.g., tissue has characteristics indicative of cancer, is a lesion, abnormal mass). Furthermore, the margins of any tissue (e.g., abnormal mass) can be identified and used in further steps, as described herein, to remove and/or treat the tissue.

Step 410 can be accomplished by reviewing the predefined characteristics identified in step 408 and selecting a procedure to treat the tissue based upon the predefined characteristics.

Step 412 can be accomplished based upon the procedure selected in step 410, the available MR scanner, and/or an MR field strength desired to complete a procedure. Any suitable medical system (e.g., first medical device and second medical device) can be selected to accomplish performance of a procedure, such as those described herein, variations of those described herein, and any other medical system and/or medical device considered suitable for a particular embodiment. Alternatively, a medical system and/or medical device can be selected from a kit, such as kit 300, or variations of the kits described herein. Each of the medical devices described herein includes a marker, or plurality of markers, that produces an image artifact, or plurality of image artifacts, under MRI that has a set of characteristics.

Step 414 can be accomplished using the medical system (e.g., first and second medical devices) selected in step 412. Alternatively, step 414 can be accomplished using any suitable medical device, such as the medical devices described herein or incorporated by reference herein. Examples of medical devices considered suitable to use to complete a method of treatment and of other steps considered suitable to include in a method of treatment are described in U.S. patent application Ser. No. 17/573,087, filed on Jan. 11, 2022, which is hereby incorporated by reference in its entirety for the purpose of describing medical devices considered suitable to complete a method of treatment and for the purpose of describing steps considered suitable to include in a method of treatment.

Step 414 can be accomplished by applying a distally-directed force on the first medical device such that a distal end of the first medical device is advanced into a bodily passage and to, within, or adjacent to, the tissue that has been identified as having the predefined characteristics. In addition, step 414 can be accomplished by applying a distally-directed force on the second medical device such that a distal end of the second medical device is advanced into a lumen defined by the first medical device and distal to the first medical device. Advancement of the second medical device can be accomplished concurrently with advancement of the first medical device, prior to advancement of the first medical device, or subsequent to advancement of the first medical device. Alternatively, the second medical device can be advanced into a lumen defined by the first medical device until the distal end of the second medical device is disposed within the lumen defined by the first medical device and between the proximal and distal ends of the first medical device.

A bodily passage can include any suitable portion of a body, including existing bodily passages, bodily lumens, and/or bodily passages created through tissues layers and/or fascia using a device described herein. Step 414 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include a first medical device and/or a second medical device, portions that include an image artifact and/or an altered image artifact, portions that include the tissue that has predefined characteristics and a first medical device and/or a second medical device, and/or any other portion of the patient considered suitable for a particular embodiment. For example, a second portion of the patient can be the same as, or different than, the first portion of the patient.

While step 414 has been described has being completed using both a first medical device and a second medical device, step 414 can be accomplished in two alternative ways. In the first alternative, a first step comprises advancing a first medical device into a bodily passage and to the tissue while scanning a second portion of the patient that includes the first medical device using the MR scanner. A second step comprises advancing the second medical device into a bodily passage and to the tissue while scanning the second portion of the patient that includes the second medical device using the MR scanner. In the second alternative, a first step comprises advancing a second medical device into a bodily passage and to the tissue while scanning a second portion of the patient that includes the second medical device using the MR scanner. A second step comprises advancing the first medical device into a bodily passage and to the tissue while scanning the second portion of the patient that includes the first medical device using the MR scanner.

An image artifact produced under MRI can include any an image artifact created by a marker included on any medical device, or combination of markers included on a combination of medical devices. For example, a first image artifact can be created by a marker disposed on a first medical device, a marker disposed on a second medical device, a combination of markers included on a first medical device and a second medical device, overlapping markers (e.g., altered image artifact), and/or any other marker considered suitable for a particular embodiment.

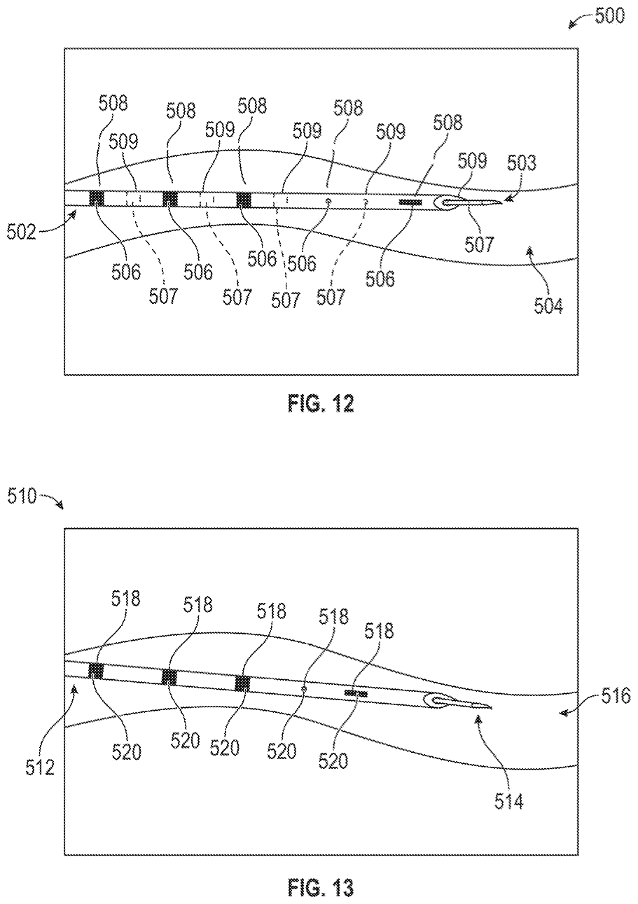

Step 416, and its alternatives, can be accomplished by obtaining the MR image from the MR scanner used in step 402 and step 414. FIG. 12 illustrates an MR image 500 showing a first medical device 502 and a second medical device 503 disposed within a bodily passage 504. The first medical device 502 includes a first plurality of markers 506. Each marker of the plurality of first markers 506 has a first magnetic susceptibility that produces an image artifact 508. The second medical device 503 includes a second plurality of markers 507. Each marker of the second plurality of markers 507 has a second magnetic susceptibility that is different than the first magnetic susceptibility and produces an image artifact 509. Each of the image artifacts 508, 509 has a set of characteristics.

Step 418 can be accomplished by reviewing the MR image obtained in step 416 and identifying the characteristics of the image artifact in the image. This can be accomplished using software (e.g., software with artifact recognition, localization, tracking, image processing, and/or machine learning) included in the MR scanner used in step 402 and/or visually by a clinician. For example, the MR scanner used in step 402, and the remainder of steps included in method 400, includes a database that correlates characteristics of image artifacts to each medical device, correlates characteristics of image artifacts of a combination of medical devices to the combination of medical devices, and/or correlates characteristics of image artifacts created by overlapping markers to a combination of medical devices, used to perform treatment such that when an image artifact is identified by the MR scanner using the characteristics of the image artifact the MR scanner can identify and/or track the medical device, or combination of medical devices, alter a first set of MR image parameters and/or sequence parameters to a second set of MR image parameters and/or sequence parameters, as described herein, and/or alter the image created by the MR scanner to create an altered MR image. The characteristics of an image artifact included in the database can include those described herein, such as the size, shape, position, orientation, pattern, and/or intensity of an image artifact. For example, each of the medical devices 10, 50, 110, 150, 210, 250, 312, 314, 316, and 318 has a distinct pattern of markers relative to the other medical devices. As a result, each of the medical devices 10, 50, 110, 150, 210, 250, 312, 314, 316, and 318, or combination of medical devices, will have distinct image artifact characteristics (e.g., pattern or code) under MRI that can be identified using the database included in the MR scanner. Image processing software or machine learning can optionally be used to determine the state of these markers, or image artifacts, in near real-time, aiding in the estimation of device positioning through automated sequence selection and allowing for path planning to targeted locations during treatment to avoid critical or sensitive anatomy.

Step 420 can be accomplished by reviewing the MR image obtained in step 416 and confirming the first medical device and the second medical device are positioned at a desired location within the bodily passage (e.g., at, within, or adjacent to, the tissue that has been identified as having the predefined characteristics). This can be accomplished by visualizing one or more image artifacts created by the markers included on the first medical device and/or second medical device, as described herein. If the first medical device and/or second medical device are/is not positioned at a desired location, an optional step comprises manipulating the position of the first medical device and/or second medical device. If the first medical device and/or the second medical device are/is not positioned at a desired location, an optional step 432 comprises repeating step 414, step 416, step 418, and step 420 to manipulate the position of the first medical device (e.g., in real time) and/or second medical device (e.g., in real time). While step 414, step 416, step 418, and step 420 have been illustrated as separate steps, these steps can optionally be completed simultaneously in real time. Optional step 432 can be accomplished any suitable number of times to correctly position a first medical device and/or second medical device.

An optional step that can be completed subsequent to step 418 and/or 420, or at any other time during the completion of method 400, comprises altering the first set of MR image parameters and/or sequence parameters (e.g., SE/GRE, TR, TE, flip angle, field-of-view, resolution, slice thickness, gradient field) to a second set of MR image parameters and/or sequence parameters based upon the identified image artifact. This optional step can be accomplished using software included in the MR scanner used in step 402 and/or a clinician. This step can be accomplished any time a MR image is taken, an image artifact is detected, an altered image artifact is detected, an image artifact is altered, multiple image artifacts are detected, or a combination of image artifacts are detected such that the MR image parameters and/or sequence parameters are regularly altered or updated based upon image artifacts produced by a marker, or a plurality of markers, disposed on a first medical device, a second medical device, and/or a combination of first and second medical devices. An altered MR image can include a superimposed graphic of the medical device (e.g., portion of medical device, entirety of medical device), or combination of medical devices, identified using the characteristics of an image artifact, or combination of image artifacts, can include an amplified, attenuated, or omitted portion of an identified medical device (e.g., distal end), combination of medical devices, image artifact, or other object within the image, and/or include any other image manipulation considered suitable for a particular embodiment. For example, an altered MR image can include a manipulation of a plane, slice, slice thickness, and/or sequence parameters based on an identified image artifact. For example, when an image artifact, or altered image artifact, is identified by the software or a clinician, an image parameter (e.g., relating to plane selection, slice, slice thickness, plan, sequence parameters) can be altered automatically (e.g., by MR scanner software) based upon the identified image artifact, or altered image artifact.

Step 422 can be accomplished by applying a force (e.g., distally-directed, proximally-directed, torque) on the first medical device and/or second medical device to alter the first image artifact (e.g., amplify, attenuate, eliminate). This can be accomplished as described herein (e.g., positioning the first and second medical devices such that a marker on the first medical device overlaps (e.g., axially) a marker on the second medical device, or positioning the first and second medical devices such that a marker on the first medical device does not overlap a marker on the second medical device). This step advantageously provides a mechanism for dynamically tuning a marker and a resulting image artifact as a procedure is completed (e.g., in real time, under continuous MR imaging), which, as described herein, can alter the way an MR scanner operates (e.g., altering image parameters and/or altering sequence parameters). For example, a clinician can alter one or more image artifacts (e.g., turn them on (e.g., amplify), turn them off (attenuate)) by manipulating the relative positions of the first and second medical devices and the markers included on the first and second medical devices.

As described herein, there is a need to leverage diagnostic imaging modalities, MR in particular, to directly guide medical therapies. As such, the identification and precise targeting of specific characteristics of tissue (e.g., lesions, abnormal anatomy) represents a significant clinical challenge. The medical systems, devices, kits, and methods described herein are intended to substantially improve upon current interventional MRI technologies by providing clinicians the ability to dynamically change the visibility of image artifacts created by markers included on a medical device throughout or across multiple procedures using passive markers. The medical systems, devices, kits, and methods described herein allow for precise identification, tracking, and navigation of interventional devices to various locations within a body and improve overall patient care (e.g., diagnosis, targeted treatment delivery, etc.) using the characteristics of the image artifacts created by a medical device, or a combination of medical devices.

For example, during at least one part of an interventional procedure, a first medical device and a second medical device can be positioned such that a marker on the first medical device overlaps a marker on the second medical device to alter (e.g., eliminate, attenuate, amplify) an image artifact of the marker on the first medical device and/or the marker on the second medical device, and the image artifacts associated characteristics. In both subtractive and additive configurations, markers that have magnetic susceptibility along the second medical device could be tailored to interact differently with markers on the first medical device based on their position or orientation with respect to the first medical device and/or the markers disposed on the first medical device. For instance, a first medical device and a second medical device could be designed with two artifact positional modes: In one, the first medical device and the second medical device are positioned such that one alternative set of image artifacts created by markers on the first medical device are canceled (or amplified) by image artifacts created by markers on the second medical device, while in the other position a different set of image artifacts of the first medical device are canceled (or amplified) by image artifacts created by markers on the second medical device. In this manner, the size, shape, and intensity of the image artifacts of the first medical device could be dynamically shifted, or changed, along the first medical device (e.g., elongate member) at any time during the procedure. Applications of these concepts extend to device tracking, following the change in a set of image artifacts as devices move relative to one another, or as a way to dynamically tune a set of image artifacts and their associated characteristics to better suit a particular part of a procedure.

Use of the medical systems, devices, kits, and methods described herein can lead to the creation of distinct image artifacts within an MR image that can be identified with software (e.g., software with artifact recognition, localization, tracking, image processing, and/or machine learning). Furthermore, as described herein, cumulative susceptibility-based artifacts that result from the combination of multiple devices could be tailored to respond differently to a single or multiple imaging sequences (gradient echo vs. spin echo) or at different magnetic field strengths. In these procedures, image processing software or machine learning can optionally be used to determine the state of these markers in near real-time, aiding in the estimation of a devices positioning through automated sequence selection and allowing for path planning to targeted locations during therapy delivery in avoidance of critical or sensitive anatomy.

Step 424 can be accomplished as described herein and by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include a first medical device and/or a second medical device, portions that include an image artifact and/or an altered image artifact, portions that include the tissue that has predefined characteristics and a first medical device and/or a second medical device, and any other portion of the patient considered suitable for a particular embodiment. For example, a third portion of the patient can be the same as, or different than, the first portion of the patient and/or the second portion of the patient.

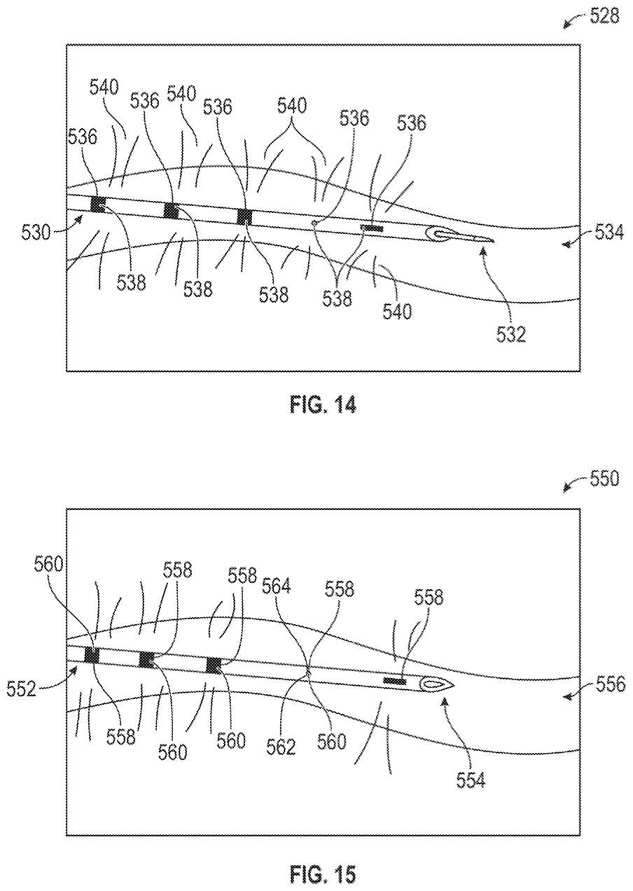

Step 426 can be accomplished by obtaining the MR image from the MR scanner used in step 402. FIG. 13 illustrates an MR image 510 showing a first medical device 512 and a second medical device 514 disposed within a bodily passage 516. The first medical device 512 includes a first plurality of markers 518 and the second medical device 514 includes a second plurality of markers 520. The overlapping arrangement of the first and second plurality of markers 518, 520 alters (e.g., amplifies, attenuates, eliminates) the image artifact produced by the first plurality of markers 518. In the embodiment shown in FIG. 13, the image artifact produced by first plurality of markers 518 and the second plurality of markers 520 are eliminated when the plurality of markers 518, 520 overlap. FIG. 14 illustrates an MR image 528 showing a first medical device 530 and a second medical device 532 disposed within a bodily passage 534. The first medical device 530 includes a first plurality of markers 536 and the second medical device 532 includes a second plurality of markers 538. The overlapping arrangement of the first and second plurality of markers 534, 538 alters (e.g., amplifies) the image artifact 540 produced by the first plurality of markers 534. FIG. 15 illustrates an MR image 550 showing a first medical device 552 and a second medical device 554 disposed within a bodily passage 556. The first medical device 552 includes a first plurality of markers 558 and the second medical device 554 includes a second plurality of markers 560. The overlapping arrangement of a marker 562 of the first plurality of markers 558 and a marker 564 of the second plurality of markers 560 alters (e.g., amplifies, attenuates, eliminates) the image artifact produced by the marker 562 of the first plurality of markers 558.

Step 428 can be accomplished using software (e.g., software with artifact recognition, localization, tracking, image processing, and/or machine learning) included in the MR scanner used in step 402 and/or a clinician. Depending on whether image parameters and/or sequence parameters have/has been updated prior to step 428, step 428 can alternatively comprise altering the second set of MR image parameters and/or sequence parameters to a third set of MR image parameters and/or sequence parameters based upon an image artifact or altered image artifact. Optionally, step 428 can be accomplished any time a MR image is taken (e.g., step 404, step 414, step 424) an image artifact is detected, an altered image artifact is detected, multiple image artifacts are detected, a combination of image artifacts are detected, and/or an image artifact is altered (e.g., step 414, step 424) such that the MR image parameters and/or sequence are regularly altered or updated based upon image artifacts produced and/or altered by a marker, or markers, disposed on first medical device, a second medical device, and/or a combination of first and second medical devices. This step can be accomplished using software included in the MR scanner used in step 602 and/or a clinician. An altered MR image can include a superimposed graphic of the medical device (e.g., portion of medical device, entirety of medical device), or combination of medical devices, identified using the characteristics of an image artifact, or combination of image artifacts, can include an amplified, attenuated, or omitted portion of an identified medical device (e.g., distal end), combination of medical devices, image artifact, or other object within the image, and/or include any other image manipulation considered suitable for a particular embodiment. For example, an altered MR image can include a manipulation of a plane, slice, slice thickness, and/or sequence parameters based on an identified image artifact. For example, when an image artifact, or altered image artifact is identified by the software or a clinician, an image parameter (e.g., relating to plane selection, slice, slice thickness, plan, sequence parameters) can be altered automatically (e.g., by MR scanner software) based upon a detected image artifact, or altered image artifact.

Interventional MR procedures generally require frequent adjustment or changes to MR imaging sequence parameters throughout various stages of a given procedure. For example, a vascular procedure may be best performed with one sequence type during device tracking (e.g., rapid frame rate, lower resolution, enhancement of certain tissue characteristics) and another sequence type once the interventional devices have been advanced to the target site (e.g., higher resolution, enhancement of different tissue characteristics to detect lesions, such as inflammation, ADC). Alternatively, one or more interventional devices may be employed across multiple MRI-based procedures, requiring specific imaging sequences to the particular device and procedure configuration. For instance, an MRI-compatible needle may be used across multiple soft-tissue biopsy procedures, each requiring different gross or fine tracking characteristics and highlighting different anatomy or tissue types. Currently, many of these imaging protocols involve manual optimization by the clinician using the MR scanner, which can introduce intra-operator variability and inefficiency.

Therefore, the medical systems, devices, kits, and methods described herein provide mechanism for at least increasing the efficiency when a procedure is completed. For example, software included on an MR scanner, such as those utilized to complete the methods described herein, can be utilized to provide automation of parameter adjustments throughout a procedure (e.g., plane selection, slice selection), or across multiple procedures, based upon the identified image artifacts created by one or more markers provided on a first medical device, one or more markers created on a second medical device, and/or alteration of image artifacts provided by overlapping markers, as described herein. Therefore, the existence of an image artifact, or artifacts, and/or dynamic changes in image artifacts that result from different device or component configurations can be used by the MR scanner or a clinician to automate the selection of MRI sequence parameters.

With some procedures, the transition from one procedural stage to another coincides with a certain configuration of two or more devices and may require a change in image protocol. Dynamic changes in marker artifacts on these devices can be tailored to create an easily identifiable image artifact (e.g., signal, change in size, shape, intensity, or pattern), or change in an image artifact, within the resulting MR image. Imaging software included on an MR scanner, such as those used to complete the methods described herein, can detect image artifacts, or change in an image artifact, and automatically optimize the MR imaging parameters and/or sequence to those best able to meet a clinician's needs for the new stage of the procedure.

The medical systems, devices, kits, and methods described herein can be used to automatically alter other imaging parameters and/or sequence within a procedure, or across multiple procedures, using the same device, or devices. For instance, changes in MRI sequence parameters (SE/GRE, TR, TE, flip angle, field-of-view, resolution, slice thickness, gradient field) can be triggered by the characteristics of the effective image artifact created by a medical device, or two or more medical devices in a particular combination that result in cumulative change in at least one image artifact. These artifact driven triggers could be used to signal transitions in device tracking, guiding how MR sequences are adapted to follow a device or component amongst a field of multiple devices, or tailor an MR sequence to highlight characteristic of a particular tissue during a specific part of a procedure (e.g., T1 v. T2 weighted images).

For example, a series of sequential MRI sequence parameters could be triggered successively based on an identified image artifact, or altered image artifact, generated by a current and/or a previous configuration of one or more devices. This process could then be used to automate imaging protocols throughout an entire procedure. Alternatively, utilizing a single base device and multiple different but compatible medical devices (e.g., various guiding or balloon catheters), all with one or more uniquely interacting susceptibility markers, the specific imaging procedure relating to the use of at least one compatible device with the base device could be automatically detected based on the identification of a unique image artifact generated by the initial multi-device configuration (e.g., combination of image artifacts created by a first medical device and a second medical device). Using the detection of this pairing, a preprogrammed set of MRI sequences could be automatically loaded and triggered by the software included on the MR scanner based on the image artifacts generated by sequential device configuration previously described. In other words, an image artifact (or series of artifacts) generated by the interaction of susceptibility markers on two or more medical devices would serve as a unique fingerprint to identify a particular medical device pairing or device configuration, with the MRI machine automatically reconfigured to the procedure, or next step in the procedure.

Step 430 can be accomplished by performing treatment using any suitable medical system and/or medical device, such as the first medical device and/or the second medical device, and/or performing the procedure selected in step 410. For example, a treatment can be performed and be based upon the tissue identified in step 408. Examples of treatments considered suitable to perform include biopsies, treatments using the medical systems and/or medical devices described herein, using a first medical device that is already disposed within a bodily passage and swapping out second medical device for a third medical device, and any other treatment considered suitable for a particular embodiment.

Each of step 404, step 406, step 408, step 410, step 412, step 414, step 416, step 418, step 420, step 422, step 424, step 426, step 428, and/or step 430 can be accomplished without removing the patient from the MR scanner within which the patient is positioned in step 402.

Method 400 is considered advantageous at least because each step of method 400 can be performed during a single patient visit and using the same MR scanner, which increases efficiency and reduces the number of patient visits and procedures performed. This results in a set of procedures in which a physician can visualize, diagnose, and treat a patient in a single patient visit. While some steps have been described as being completed while scanning a portion of the patient using a MR scanner and other steps have not been described as being performed while scanning a portion of the patient using a MR scanner, any step described herein can be completed while scanning a portion of a patient using the MR scanner, and/or an ultrasound device or without scanning a portion of a patient using a MR scanner. In embodiments in which an ultrasound image is obtained, the MR image obtained can be electronically fused with a real-time ultrasound image (e.g., transrectal ultrasound image of a prostate). While some steps have been described as being completed while scanning a portion of the patient using a MR scanner, this step can be broken into two separate steps such that a subsequent step of scanning a portion of a patient using the MR scanner can be accomplished. Furthermore, any step which is completed while scanning a portion of the patient using the MR scanner can comprise obtaining a single still image and be repeated any desired number of times to obtain multiple MR images that can be grouped as a cine to show motion and/or any step which is completed while scanning a portion of the patient using the MR scanner can comprise obtaining a live image, such as being completed under live real-time MRI visualization.

The medical systems, devices, kits, and methods described herein advantageously provide susceptibility-based maker artifacts that can be dynamically changed throughout a procedure, or across multiple procedures, using a system, or set of devices, components, or features in different configurations. As such, the medical systems, devices, kits, and methods described herein expand upon the current use of fixed susceptibility marker artifacts and increase the versatility of medical device use in MR-guided interventional procedures. Additionally, by exploiting the position based additive and subtractive properties of susceptibility marker magnetic field distortions (e.g., superposition), the medical systems, devices, kits, and methods described herein can be utilized on a variety of medical devices that can be used in combination with one another. Finally, the medical systems, devices, kits, and methods described herein can replicate, or at least mimic, aspects of dynamically tunable image artifacts generated by semi-active resonant circuits or active antenna coils, but without the need for circuit integration or external power sources.

An interventional system with dynamically-tunable passive markers, capable of real-time adjustments in image artifact size, shape, or intensity throughout a procedure, or across multiple procedures, increases the efficiency associated with patient care and improves upon the current medical systems considered suitable for use with MRI-guided treatments. For example, the medical systems, devices, kits, and methods described herein simplify the clinician experience during an MRI-guided procedure. This includes automatically recognizing marker image artifacts or changes, interpreting new marker image artifacts as representative of a new stage within a procedure or as a new procedure, and changing or optimizing the MRI sequence parameters to those best able to meet the clinician's needs at a given phase or stage of the procedure based upon the identified image artifacts. This removes the need for manual sequence adjustment, standardizes the procedures and imaging parameters, and improves the safety and efficiency of interventional MR procedures.

While a number of methods have been described herein, it will be appreciated that a method may be a non-invasive method that does not require an invasive intervention by a medical professional. For example, a method may be carried out within a body lumen or passageway, such as the ear canal or a nasal passage, for example in order to place a device within such a passageway. Equally, methods may be implemented on a cadaver or artificial body parts for example for training purposes. Moreover, the skilled person will appreciate that the methods described herein may not be used on the human or animal body at all, but may be used in order to view other types of devices using MRI imaging techniques, for example in an industrial setting.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular arrangement of elements and steps disclosed herein have been selected by the inventor(s) simply to describe and illustrate examples of the invention and are not intended to limit the scope of the invention or its protection, which is to be given the full breadth of the appended claims and any and all equivalents thereof.

What is claimed is:

1. A medical system useful in performing treatment under magnetic resonance imaging comprising:
a first medical device having a first passive marker, the first passive marker formed of a first material having a first magnetic susceptibility; and
a second medical device having a second passive marker, the second passive marker formed of a second material having a second magnetic susceptibility, the second passive marker creating an image artifact under MRI;
wherein the image artifact created by the second passive marker is altered under MRI when the first passive marker overlaps the second passive marker.

2. The medical system of claim 1, wherein the first magnetic susceptibility is the same as the second magnetic susceptibility.

3. The medical system of claim 2, wherein the image artifact created by the second passive marker is amplified when the first passive marker overlaps the second passive marker.

4. The medical system of claim 1, wherein the first magnetic susceptibility is different than the second magnetic susceptibility.

5. The medical system of claim 4, wherein the image artifact created by the second passive marker is attenuated when the first passive marker overlaps the second passive marker.

6. The medical system of claim 1, wherein the first material is the same as the second material.

7. The medical system of claim 1, wherein the first material is different than the second material.

8. The medical system of claim 1, wherein the first medical device comprises an elongate member, the first passive marker is disposed on the elongate member of the first medical device; and
wherein the second medical device comprises an elongate member, the second passive marker is disposed on the elongate member of the second medical device.

9. The medical system of claim 8, wherein the elongate member of the first medical device is formed of a third material that is different than the first material.

10. The medical system of claim 9, wherein the elongate member of the second medical device is formed of a fourth material that is different than the second material.

11. The medical system of claim 10, wherein the third material and the fourth material are titanium.

12. The medical system of claim 8, wherein the first medical device and the second medical device can be used coaxially.

13. The medical system of claim 1, wherein the first passive marker comprises a first plurality of passive markers, each marker in the first plurality of passive markers formed of the first material, each marker in the first plurality of passive markers creating an image artifact under MRI;
wherein the second passive marker comprises a second plurality of passive markers, each marker in the second plurality of passive markers formed of the second material, each marker in the second plurality of passive markers creating an image artifact under MRI; and
wherein the image artifact created by each marker in the second plurality of passive markers is altered when the first plurality of passive markers overlaps the second plurality of passive markers.

14. The medical system of claim 1, wherein the first passive marker comprises a first plurality of passive markers, the first plurality of passive markers including a first subset of markers, a second subset of markers, and a third subset of markers, each marker in the first subset of markers formed of the first material, each marker in the second subset of markers formed of a third material having a third magnetic susceptibility, each marker in the third subset of markers formed of a fourth material having a fourth magnetic susceptibility, the first magnetic susceptibility being different than the third magnetic susceptibility and the fourth magnetic susceptibility, the third magnetic susceptibility being different than the fourth magnetic susceptibility.

15. The medical system of claim 14, wherein the second passive marker comprises a second plurality of passive markers, the second plurality of passive markers including a first subset of markers, a second subset of markers, and a third subset of markers, each marker in the first subset of markers of the second plurality of passive markers formed of the second material, each marker in the second subset of markers of the second plurality of passive markers formed of a fifth material having a fifth magnetic susceptibility, each marker in the third subset of markers of the second plurality of passive markers formed of a sixth material having a sixth magnetic susceptibility, the second magnetic susceptibility being different than the fifth magnetic susceptibility and the sixth magnetic susceptibility, the fifth magnetic susceptibility being different than the sixth magnetic susceptibility; and
wherein each marker in the first subset of markers of the second plurality of markers creates an image artifact under MRI, each marker in the second subset of markers of the second plurality of markers creates an image artifact under MRI, and each marker in the third subset of markers of the second plurality of markers creates an image artifact under MRI.

16. The medical system of claim 15, wherein the image artifact created by each marker in the first subset of markers of the second plurality of passive markers is altered when the first subset of markers of the first plurality of passive markers overlaps the first subset of markers in the second plurality of passive markers;
wherein the image artifact created by each marker in the second subset of markers of the second plurality of passive markers is altered when the second subset of markers of the first plurality of passive markers overlaps the second subset of markers in the second plurality of passive markers; and
wherein the image artifact created by each marker in the third subset of markers of the second plurality of passive markers is altered when the third subset of markers of the first plurality of passive markers overlaps the third subset of markers in the second plurality of passive markers.

17. A method of performing treatment under magnetic resonance imaging comprising:
positioning a patient within a magnetic resonance scanner;
scanning a first portion of the patient using the magnetic resonance scanner, the magnetic resonance scanner having a first set of MR image parameters;
obtaining a magnetic resonance image of the first portion of the patient;
identifying a tissue that has predefined characteristics using the magnetic resonance image;
selecting a procedure to treat the tissue based upon the predefined characteristics;
selecting a medical system comprising a first medical device and a second medical device to accomplish performance of the procedure;
while the patient is positioned within the magnetic resonance scanner used to scan the first portion of the patient, advancing the medical system into a bodily passage and to the tissue while scanning a second portion of the patient that includes the medical system and a first image artifact using the magnetic resonance scanner;
obtaining a magnetic resonance image of the second portion of the patient that includes the medical system and the first image artifact;
identifying the first image artifact within the MR image;
confirming the position of the medical system within the bodily passage;
manipulating the position of the first medical device relative to the second medical device to alter the first image artifact to create an altered image artifact;
scanning a third portion of the patient that includes the medical system and the altered image artifact using the magnetic resonance scanner;
obtaining a magnetic resonance image of the third portion of the patient that includes the medical system and the altered image artifact;
altering the first set of MR image parameters to a second set of MR image parameters based upon the altered image artifact; and
performing treatment.

18. The method of claim 17, wherein the first medical device has a first marker, the first marker is a passive marker formed of a first material, the first material having a first magnetic susceptibility; and
wherein the second medical device has a second marker, the second marker is a passive marker formed of a second material, the second material having a second magnetic susceptibility.

19. The method of claim 18, wherein the first magnetic susceptibility is the same as the second magnetic susceptibility.

20. A method of performing treatment under magnetic resonance imaging comprising:
positioning a patient within a magnetic resonance scanner;
scanning a first portion of the patient using the magnetic resonance scanner, the magnetic resonance scanner having a first set of MR image parameters;
obtaining a magnetic resonance image of the first portion of the patient;
identifying a tissue that has predefined characteristics using the magnetic resonance image;
selecting a procedure to treat the tissue based upon the predefined characteristics;
selecting a first medical device to accomplish performance of the procedure, the first medical device having a first marker;
while the patient is positioned within the magnetic resonance scanner used to scan the first portion of the patient, advancing the first medical device into a bodily passage and to the tissue while scanning a second portion of the patient that includes a first image artifact created by the first marker, the first image artifact having a first set of characteristics;
obtaining a magnetic resonance image of the second portion of the patient that includes the first image artifact;
identifying the first image artifact within the MR image using the first set of characteristics;
altering the first set of MR image parameters to a second set of MR image parameters based upon the identified first image artifact; and
performing treatment.
